(19) United States
(12) Patent Application Publication
(10) Pub. No.: US 2023/0129848 A1
(43) Pub. Date: Apr. 27, 2023

(54) TARGET DELIVERY OF CHEMICAL TRACERS FOR SINGLE WELL CHEMICAL TRACER TESTS

(71) Applicant: SAUDI ARABIAN OIL COMPANY, Dhahran (SA)

(72) Inventors: Nan Shi, Damman (SA); Jesus Manuel Felix Servin, Dhahran (SA); Amr I. Abdel-Fattah, Dhahran (SA)

(73) Assignee: SAUDI ARABIAN OIL COMPANY, Dhahran (SA)

(21) Appl. No.: 17/451,706

(22) Filed: Oct. 21, 2021

(51) Int. Cl. E21B 47/11; C09K 8/035

(52) U.S. Cl. CPC E21B 47/11; C09K 8/035; B82Y 30/00; C09K 2208/10

(57) ABSTRACT

A single well chemical tracer composition comprising core/shell tracer particles and an aqueous fluid is provided. The core/shell tracer particles have a core and a polymer shell. At least two tracer chemicals are encapsulated in the core/shell tracer particles. A method of determining residual oil in a reservoir is also provided. The method includes introducing a tracer fluid having the core/shell tracer particles into a wellbore. The reservoir is then maintained for a period of time such that the core/shell particle is ruptured, and the tracer chemicals are released into the reservoir. Then, the method includes producing a produced fluid from the reservoir, measuring the quantity of the tracer chemicals in the produced fluid, and determining a residual oil content of the reservoir based on the measured quantity of the tracer chemicals in the produced fluid.

### BACKGROUND

During primary oil recovery, oil inside an underground hydrocarbon reservoir is driven to the surface (for example, toward the surface of an oil well) by a pressure difference between the reservoir and the surface. However, only a fraction of the oil in an underground hydrocarbon reservoir can be extracted using primary oil recovery. Thus, a variety of techniques for enhanced oil recovery are utilized after primary oil recovery to increase the production of hydrocarbons from hydrocarbon-bearing formations. Some examples of these techniques include water flooding, chemical flooding, and supercritical CO₂ injections.

Even after primary oil recovery and enhanced oil recovery methods, significant oil may remain in a reservoir. Techniques for measuring the quantity of residual oil in a reservoir are useful for reservoir management and evaluation of the performance of enhanced oil recovery techniques. One such technique is a single well chemical tracer test (“SWCTT”). A SWCTT begins with the injection of a first tracer, followed by a pushing bank that contains a second tracer. The first tracer undergoes hydrolysis during well shut-in to generate a third product tracer. The first and third tracers are selected to have different partitioning constants so that the difference in their breakthrough curves informs the value of residual oil in the reservoir. Finally, all tracers are produced back at the surface and their concentrations measured at specific produced volume. The breakthrough curves of these three components may be used to determine residual oil in the well.

### SUMMARY

This summary is provided to introduce a selection of concepts that are further described in the detailed description. This summary is not intended to identify key or essential features of the claimed subject matter, nor is it intended to be used as an aid in limiting the scope of the claimed subject matter.

In one aspect, embodiments disclosed herein relate to a single well chemical tracer composition comprising core/shell tracer particles and an aqueous fluid. The core/shell tracer particles have a core and a polymer shell. At least two tracer chemicals are encapsulated in the core/shell tracer particles.

In another aspect, embodiments disclosed herein relate to a method of determining residual oil in a reservoir. The method includes introducing a tracer fluid into a wellbore. The tracer fluid comprises core/shell tracer particles having a porous core and a polymer shell, and at least two tracer chemicals are encapsulated in the core/shell tracer particles. The reservoir is then maintained for a period of time such that the core/shell particle is ruptured, and the tracer chemicals are released into the reservoir. Then, the method includes producing a produced fluid from the reservoir, measuring the quantity of the tracer chemicals in the produced fluid, and determining a residual oil content of the reservoir based on the measured quantity of the tracer chemicals in the produced fluid.

Other aspects and advantages of the claimed subject matter will be apparent from the following description and the appended claims.

### BRIEF DESCRIPTION OF DRAWINGS

FIG. 1A is a depiction of chemical tracer particle in accordance with one or more embodiments.

FIG. 1B is a depiction of chemical tracer particle in accordance with one or more embodiments.

FIG. 2 is a method of making a chemical tracer composition in accordance with one or more embodiments.

FIG. 3 is a depiction of well environment in accordance with one or more embodiments.

FIG. 4 is a method of determining residual oil in a well in accordance with one or more embodiments.

FIG. 5A is a depiction of chemical tracer particles in a well environment in accordance with one or more embodiments.

FIG. 5B is a depiction of chemical tracer particles in a well environment in accordance with one or more embodiments.

### DETAILED DESCRIPTION

Single well chemical tracer tests may be used to determine residual oil in a single well. In a single well chemical tracer test, a first tracer chemical, typically an ester, is injected into the well. The ester reacts with water downhole to produce a product tracer. A second tracer chemical is introduced that does not react downhole. After the tracers are introduced, the well is shut-in to ensure enough of the product tracer is generated. After the shut-in period, the tracers are produced back and the ratio of the tracer components in produced fluid may be used to determine residual oil in the well. However, hydrolysis of the first tracer can be slow, resulting in an artificially low amount of the product tracer detected at the surface or extended shut-in periods to ensure a significant amount of the product tracer is generated. Furthermore, the tracer chemicals readily diffuse through the well, which flattens the breakthrough curve and can make concentration measurements less accurate.

In a single well chemical tracer test, the estimate of residual oil saturation depends on effluent concentrations of the reactive and product tracers. The selection of the right injection concentration of the reactive tracer is thus important to generate a detectable amount of the product tracer and to avoid an overuse of the reactive tracer. Often, the use of tracers for saturation measurement depends on chromatographic retardation of two tracers, one is soluble in both water and oil and another only soluble in water. Thereby, when transported a given distance, the two tracers exhibit different times of flight.

In a single well application, one of those two tracers is generated in situ. This is done by injecting a reacting tracer, which is soluble in both water and oil. After the reacting tracer is pushed to the desired depth of investigation, the well is shut-in for few days to allow hydrolysis of the reacting tracer. Upon hydrolysis, this reacting tracer yields another (product) tracer that is only soluble in water.

In this way, before back production the two tracers are located at the same distance from the well. When the well is opened for production, the reacting tracer lags relative to the product tracer. This is due to the partitioning of the reacting tracer between the mobile aqueous phase and the stationary residual oleic phase. Thus, through monitoring effluent concentrations of the reacting and product tracers, the residual oil saturation can be determined from the time lag of the two peaks or from the shape of the breakthrough curves via curve fitting techniques.

In exemplary embodiments the tracers used in single well tracing are the alkyl esters of fatty acids and alcohols. The ester selection depends on the specific reservoir characteristics, as discussed below. One commonly used ester is ethyl acetate which hydrolyzes and forms ethylic alcohol (a new tracer) and acetic acid. The tracer (the ester) is injected into the reservoir, in a single well, up to a certain distance depending on the injected tracer quantity. The well is then shut in to allow the ester to react with reservoir water and get partly hydrolyzed. This results in the presence of a new tracer in the reservoir. The new tracer, together with the non-hydrolyzed ester, forms a pair of tracers at a certain distance in the reservoir. The well starts again to produce and both tracers are detected in the produced fluids. Because they have differences in water and in oil distribution coefficients (the product tracer is only soluble in water while the reacting tracer is soluble in both water and oil), they will reach the wellbore at different times. This time difference and the differences in their breakthrough curves are the basis in calculating the residual oil saturation around the wellbore.

The present disclosure relates to compositions and methods related to a single well chemical tracer test to determine residual oil in a well. The disclosed composition includes a core/shell particle having encapsulated chemical tracers. The core/shell tracer particles may be introduced into the well in a single step, and may be controllably released upon rupture of the shell material downhole. The disclosed compositions may lead to sharper breakthrough curves, more accurate data, and simplified procedures, as compared to conventional methods.

### Single Well Chemical Tracer Composition

In one or more embodiments, the present disclosure relates to a single well chemical tracer composition. One or more embodiments of the disclosed composition includes an aqueous fluid and a core/shell particle having at least two encapsulated tracer chemicals.

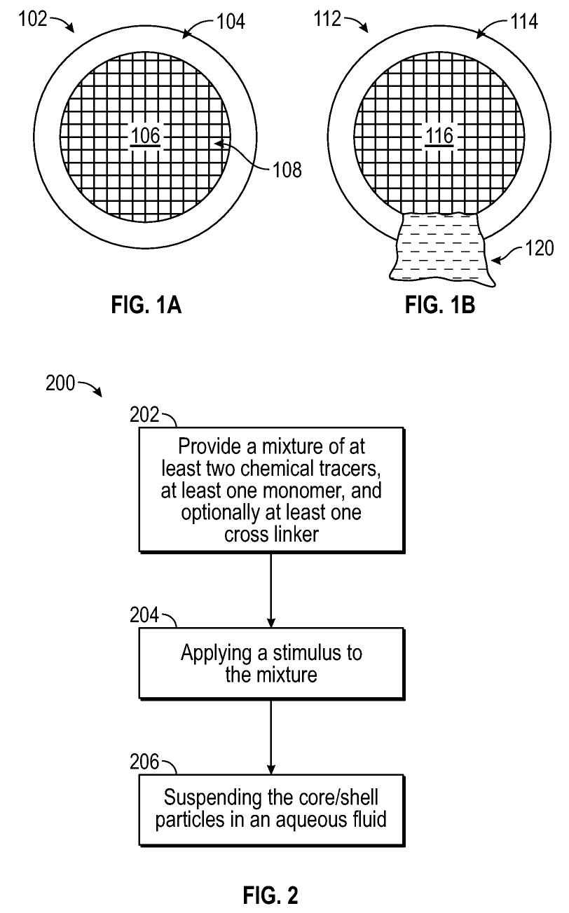

A simplified depiction of a core/shell particle in accordance with the present disclosure is shown in FIG. 1A. The particle 102 has a shell 104 and a core 106. The shell is a coating on the outer surface of the core. The shell essentially covers the entire surface of the core particle. The core 106 is comprised of a nanoparticle having pores 108 such that the core of the particle includes void space(s). The simplified schematic in FIG. 1A depicts pores having a uniform size and shape, however, the size and shape of the pores are not particularly limited and may be varied and/or irregular. The core 106 may include tracer chemicals within the pores 108 (not shown). The tracer chemicals are encapsulated in the core/shell particle by the shell 104, meaning the tracer chemicals remain inside the nanoparticle so long as the shell 104 is intact. In other embodiments, the core 106 may simply be composed of liquid tracer chemicals encapsulated by a shell 104.

As will be explained in greater detail below, core/shell particles in accordance with the present disclosure may rupture to release chemical tracers encapsulated in the nanoparticle. A ruptured core/shell particle 112 is shown in FIG. 1B. As used herein, “rupture” means that the shell of the core/shell particle has been altered such that the shell material no longer completely coats the surface of the particle. A rupture may include alteration to only a portion of the shell. When the shell 114 has been ruptured, encapsulated chemical tracer 120 may be released from the core 116 of the particle 112 into the surrounding environment.

The core/shell particle may include a porous core. Suitable core materials are those that include a porosity sufficiently high to encapsulate the tracer chemicals. The core material may be organic, such as a polymer, or inorganic. Examples of suitable polymer core materials include, but are not limited to, poly(methylmethacrylate) (PMMA), poly(ethylcyanoacrylate) (PECA), and poly(butylcyanoacrylate). Examples of suitable inorganic core materials include, but are not limited to, aluminosilicates, such as zeolites, silicates, such as MCM-41, and silicas.

As previously described, the core/shell particle includes pores suitable for storing tracer chemicals. In order to accommodate the disclosed tracer chemicals, in one or more embodiments, the core of the core/shell particle may have a surface area of at least 100 m²/g (meters squared per gram). The pores of the core material may have a suitable pore size for storing encapsulated tracer chemicals. In one or more embodiments, the average pore size of the pores in the nanoparticle core may be from about one nanometer (nm) to about 50 nm. The nanoparticle core of the core/shell particle may be a suitable size for use in a single well chemical tracer test. In particular, the average particle size of the nanoparticle core may be less than one micron, allowing for transport of the core/shell particles through the porous formation.

As previously described, in one or more embodiments, the core/shell particle includes at least two tracer chemicals suitable for a single well chemical tracer test. As used herein, “chemical tracer” may be used interchangeably with “tracer chemical.” One type of chemical tracer used in hydrocarbon-bearing formations for determining residual oil are “partitioning” tracers. A partitioning tracer is a chemical that is soluble in both oil and water, and may reach an equilibrium concentration in both phases. The extent to which these tracers are soluble in oil as compared to water may be described using a partitioning coefficient. The partitioning coefficient is the ratio between the concentration of the chemical in the water phase and the oil phase (kₚ = Cₒᵢₗ/C_{water}). The partition coefficient may be measured directly by measuring the concentration of the component in each of the two phases in an equilibrium system.

The partitioning coefficient (K value) defines the ratio of reactive tracer concentrations in the oil and water phases at equilibrium. It depends on the oil composition, injection water chemistry and reservoir temperature. A reactive tracer K value is measured in the laboratory through batch experiments and at multiple concentrations to ensure a relatively constant value over the range of concentrations expected through the test. A tracer that partitions strongly into the oil phase (i.e., large partitioning coefficient) would prolong the test duration. On the other hand, a tracer with a small partitioning coefficient makes discerning the differences in mean residence times difficult. If the residual oil saturation is expected to be high, a tracer with a low K value can be selected and the test can be terminated earlier. If the residual oil saturation is low, a low K value tracer will not exhibit sufficient retardation for a unique estimate of residual oil. An exemplary equation for determining a suitable tracer is given by:

$$0.2 \left(1 - {\text{S}}_{\text{or}}\right) / {\text{S}}_{\text{or}} < \text{K} < 3 \left(1 - {\text{S}}_{\text{or}}\right) / {\text{S}}_{\text{or}}$$

where Sₒᵣ is the expected residual oil saturation.

In a single well chemical tracer test, tracer chemicals with distinct partitioning dynamics are used to determine the amount of residual oil in the well. At least two tracer chemicals are used, each having a different partitioning coefficient. The partitioning coefficients of various tracer chemicals vary depending on a variety of factors including the oil composition in the reservoir, the reservoir temperature, and the injection water chemistry. Examples of suitable chemical tracers include, but are not limited to, ethyl acetate, ethanol, n-propyl alcohol and isopropyl alcohol.

In one or more embodiments, the disclosed core/shell particles may include a suitable amount of encapsulated tracer chemicals. In some embodiments, the core/shell particle may include up to 50 wt.% (weight percent), up to 55 wt.%, up to 60 wt.%, up to 65 wt.%, or up to 70 wt.% of at least two tracer chemicals based on the total mass of the core/shell particle.

As previously described, core/shell particles in accordance with the present disclosure include a shell surrounding the core nanoparticle. In one or more embodiments, the shell of the core/shell particle may be a polymer shell. Suitable polymer materials include polymers that are stable under room temperature and pressure conditions, and physically or chemically degrade under formation conditions. Such conditions may include elevated temperature, elevated pressure, a change in pH, increased salinity, and combinations thereof.

Suitable types of polymers for use as a shell material include, but are not limited to, polyesters and polyamides. Polymers used as the shell material in accordance with the present disclosure may have linear or branched structures and in some embodiments, may be crosslinked.

Polymers for use as a shell material may be made from suitable monomers, such as methyl methacrylate, ethyl cyanoacrylate, and butyl cyanoacrylate.

In one or more embodiments, polymers for use as a shell material are synthesized using crosslinkers in addition to monomers. In such embodiments, the crosslinkers may be selected such that they can be readily exchanged with ionic species in the reservoir fluids. This may facilitate rupture of the polymer shell downhole to release encapsulated tracer. Examples of suitable crosslinkers include but are not limited to (1,4-phenylene)bis(acrylamide); N,N′-bis(acryloyl)cystamine; 2-(methacryloylamino)ethyl 2-methyl acrylate; and N,N′-methylenebisacrylamide.

Polymer shells in accordance with one or more embodiments have a thickness that may be tuned to produce a rupture of the polymer shell at a desired location within the formation. As may be appreciated by those skilled in the art, a core/shell particle with a thinner polymer shell may rupture more easily than one with a thicker polymer shell. As such, depending upon formation conditions, the polymer shell may be appropriately designed to release the chemical tracers at an appropriate time and in the appropriate location in the formation.

In one or more embodiments, the polymer shell may have a thickness of from about 1 nm to about 50 nm. The polymer shell thickness may have a lower limit of one of 1, 2, 5, 10, 15, 20 and 25 nm and an upper limit of one of 30, 40, 45 and 50 nm, where any lower limit may be paired with any upper limit.

The polymer shell may have an appropriate surface chemistry for stability in a given chemical system, attraction or repulsion from rock surfaces in the reservoir, or diffusion/transport through the reservoir. In one or more embodiments, the polymer shell surface may be hydrophobic.

As previously described, tracer compositions in accordance with the present disclosure include an aqueous fluid. The aqueous fluid is provided to disperse the core/shell tracer particles for injection into the well. The aqueous fluid includes water. The water may be distilled water, deionized water, tap water, fresh water from surface or subsurface sources, production water, formation water, natural and synthetic brines, brackish water, natural and synthetic sea water, black water, brown water, gray water, blue water, potable water, non-potable water, other waters, and combinations thereof, that are suitable for use in a wellbore environment. In one or more embodiments, the water used may naturally contain contaminants, such as salts, ions, minerals, organics, and combinations thereof, as long as the contaminants do not interfere with the chemical tracer operations. In one or more embodiments, viscosifiers may be added to the aqueous fluid to enhance the dispersion stability of the tracers in the fluid.

The disclosed chemical tracer composition includes a suitable concentration of the core/shell tracer particles to deliver the chemical tracers to the formation. The concentration of core/shell particles in the composition should be kept suitably low such that the injectability of the fluid is not hindered. In one or more embodiments, the disclosed composition may include from about 1.0 to 10.0 vol.% (volume percent) of the core/shell particles based on the total volume of the chemical tracer composition. The concentration of the core/shell particles may have a lower limit of one of 1.0, 2.0, 3.0. 4.0 and 5.0 vol.% and an upper limit of one of 6.0, 7.0, 8.0, 9.0 and 10.0 vol.% based on the total volume of the chemical tracer composition, where any lower limit may be paired with any mathematically compatible upper limit.

### Method of Making A Chemical Tracer Composition

One or more embodiments of the present disclosure relate to a method of making the previously described single well chemical tracer composition. An exemplary method 200 is shown in FIG. 2. The method may include providing a mixture of at least two chemical tracers, at least one monomer, and optionally at least one crosslinker 202. The chemical tracers, optional cross linkers, and monomers may be appropriately selected based upon the core/shell tracer particle that is being made.

The method 200 may then include applying a stimulus to the mixture 204. The stimulus may be any stimulus suitable for polymerizing the monomers and cross linkers in the mixture. The stimulus may include, but is not limited to, UV radiation, heat, and chemical stimuli. The application of the stimulus polymerizes the monomers in the mixture, resulting in a core/shell particle encapsulating the tracer chemicals.

The method 200 may then include suspending the resultant core/shell particles in an aqueous fluid 206 to make a chemical tracer composition in accordance with one or more embodiments.

### Method of Using a Chemical Tracer Composition

One or more embodiments of the present disclosure relate to a method of using the previously described single well chemical tracer composition to determine residual oil in a single well environment.

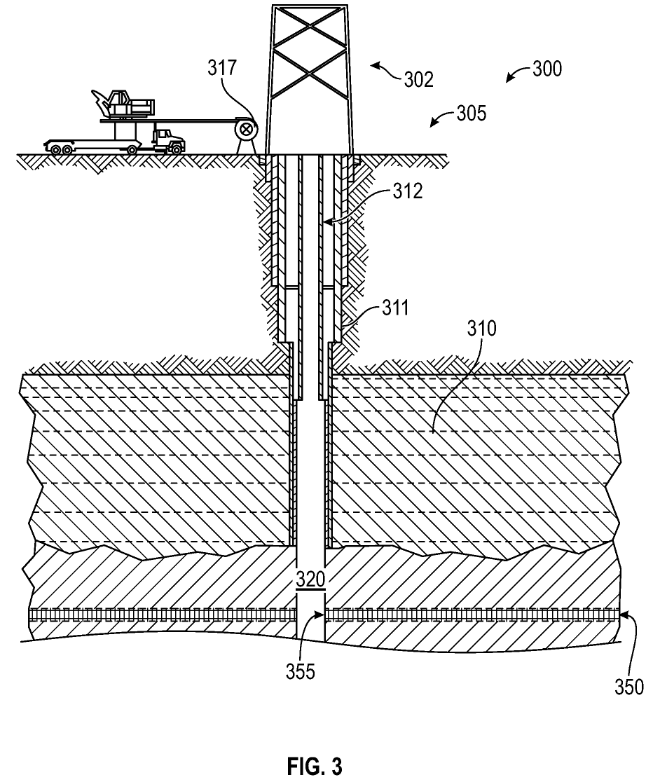

FIG. 3 is a diagram that illustrates a well environment 300 in accordance with one or more embodiments. Well environment 300 includes a subsurface 310. Subsurface 310 is depicted having a wellbore wall 311 both extending downhole from a surface 305 into the subsurface 310 and defining a wellbore 320. The well environment includes a well head 302 at the surface 305. The subsurface also includes target formation 350 in which residual oil is determined. Target formation 350 has target formation face 355 that fluidly couples target formation 350 with wellbore 320 through wellbore wall 311. In this case, casing 312 extends downhole through the wellbore 320 into the subsurface 310 and towards target formation 350.

With the configuration in FIG. 3, the previously described single well chemical tracer composition may be introduced into the subsurface 310 and towards target formation 350 via a pump 317 through valves located in the well head. Hydrocarbon-bearing formations may include any oleaginous fluid, such as crude oil, dry gas, wet gas, gas condensates, light hydrocarbon liquids, tars, and asphalts, and other hydrocarbon materials. Hydrocarbon-bearing formations may also include aqueous fluid, such as water and brines. Hydrocarbon-bearing formations may include formations with pores sizes of from about 100 nm to 100 µm. As such, embodiment core/shell tracer particles have sizes in an appropriate range to traverse pores of hydrocarbon-bearing formations. Embodiment chemical tracer compositions may be appropriate for use in different types of subterranean formations, such as carbonate, shale, sandstone and tar sands.

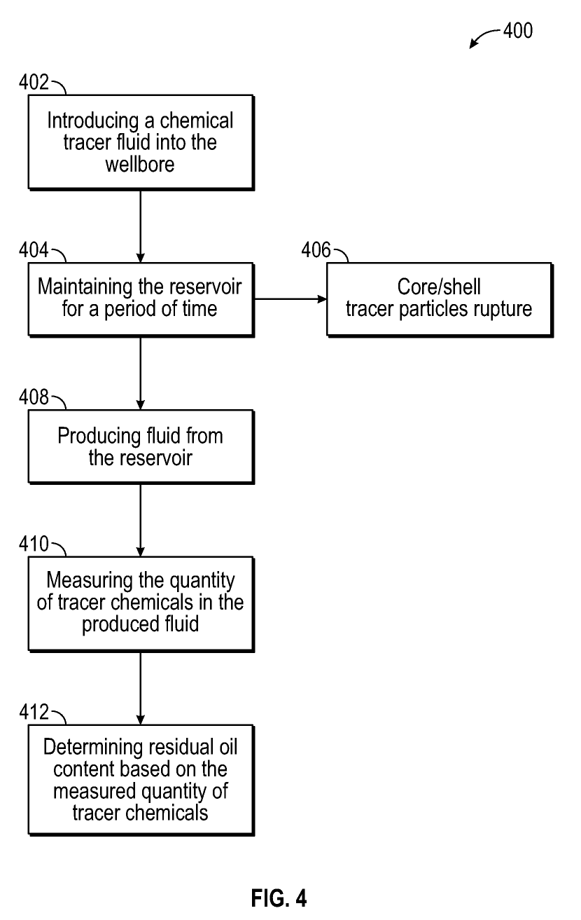

A method of determining residual oil in a reservoir in accordance with one or more embodiments of the present disclosure is shown in FIG. 4. The method 400 includes introducing a tracer fluid into a wellbore 402. The tracer fluid includes the previously described core/shell tracer particles with at least two tracer chemicals. In one or more embodiments, prior to introducing the residual oil into the reservoir, the target formation 350 may be flushed of mobile oil and other residual chemicals using water. In particular embodiments, 2 to 3 pore volumes of fluid are injected to flush mobile oil from the target formation. After flushing, the well is produced to ensure that no oil is mobile. When this condition is met, the tracer may be introduced into the well.

The method 400 may then include maintaining the reservoir for a period of time 404. This step of maintaining may also be referred to as the “shut-in” step. In this period of time, fluid is not produced from the well. During the shut-in step, the core/shell particle is ruptured 406, releasing the chemical tracer(s) into the reservoir. Once the tracers are released, they dissolve into the oil and water phases in the well and reach their respective equilibrium concentrations based on their partitioning coefficients. The shut-in step occurs for several hours up to one day when the disclosed core/shell particles are employed. This provides a substantial improvement over conventional tracers, which typically a shut-in period of 2-3 days.

The method 400 may then include producing a produced fluid from the reservoir 408. As a result of the previously described shut-in step, the produced fluid contains the previously described chemical tracer(s). An amount of the produced fluid may be collected for analysis. The produced fluid is produced from the reservoir 408 until the total produced fluid is at least three pore volumes or when the tails of the concentration profile of the chemical tracers are reached.

The method 400 may then include measuring the quantity of the chemical tracer(s) in the produced fluid 410. The concentration of each tracer may be measured on-site via spectroscopic techniques such as gas-chromatography mass-spectrometry (GC-MS), high-pressure liquid chromatography (HPLC), optical spectroscopy (e.g. UV-vis), and nuclear magnetic resonance (NMR).

The method 400 may then include determining a residual oil content of the reservoir based on the measured quantity of the chemical tracer(s) in the produced fluid 412. The residual oil may be determined using methods known by those skilled in the art. As fluid is produced from the well and the concentration of each tracer is measured, the relative concentrations of the tracers having different partitioning coefficients is used to determine the residual oil in the well.

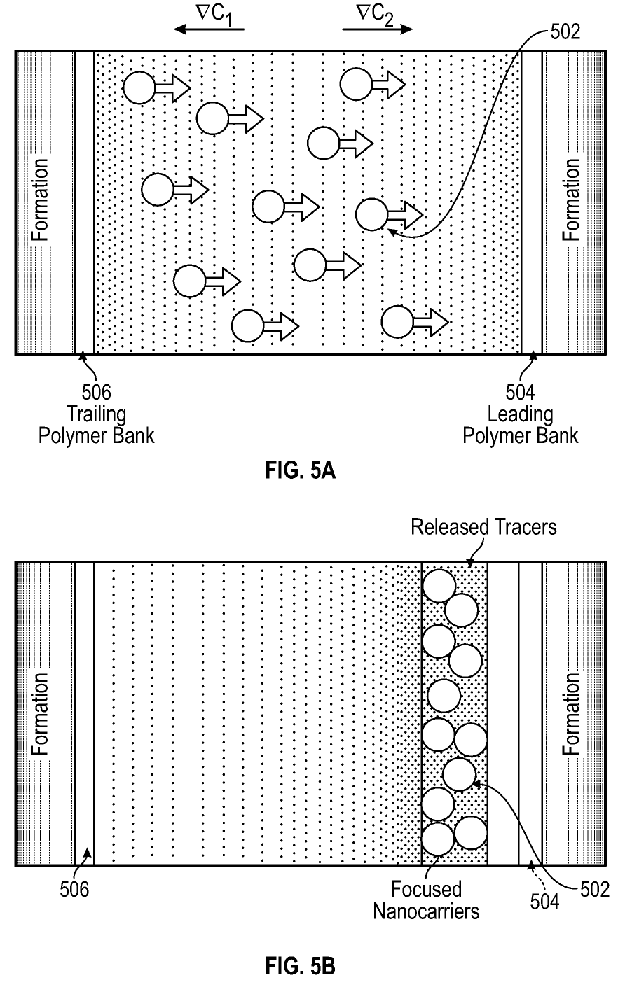

In one or more embodiments, the methods disclosed herein may include additional steps that may improve the accuracy of the residual oil information collected during the chemical tracer process. In such embodiments, a chemical gradient may be created in the formation to focus the chemical tracers in a certain area of the well. A schematic of the process of creating a chemical gradient to produce a focused band of tracer particles is shown in FIGS. 5A and 5B.

Referring to FIG. 5A, a first polymer fluid 504 may be introduced into the well. The first polymer fluid may also be referred to as “the leading polymer bank.” The leading polymer bank has a first chemistry. The first chemistry may be a concentration of a certain component or a charge of a certain component. Suitable polymers are water soluble and stable under reservoir conditions such as high salinity, high temperature and high pressure. Additionally, polymers that provide improved viscosity are also suitable. For example, the first polymer fluid may have a first concentration of a polymer. Polymers suitable for use in oil and gas operations, such as Flopamm™ AN-132 and Flopamm™ AN-125 available from SNF, for example, may be used to create the first polymer fluid.

After the introduction of the leading polymer bank into the well, the core/shell tracer particles 502 are introduced into the well. Once the core/shell tracer particles have been introduced into the well, a second polymer fluid 506 having a second chemistry is introduced into the well. The second polymer fluid may also be referred to as “the trailing polymer bank.” Similar to the first polymer fluid, suitable polymers are water soluble and stable under reservoir conditions such as high salinity, high temperature and high pressure. Additionally, polymers that provide improved viscosity are also suitable.

The second chemistry is different from the first chemistry. The second chemistry may be a concentration of a certain component or a charge of a certain component, so long as the charge or concentration of the second fluid is different from the charge or concentration of the first fluid. For example, the second polymer fluid may include the same polymer as was used in the first polymer fluid, however the second polymer fluid may have a different concentration of the polymer. Similarly, the second polymer fluid may include a polymer that as a different charge as compared to the polymer used in the first polymer fluid. In one or more embodiments, the polymer included in the second polymer fluid may be a polymer suitable for use in oil and gas applications, such as, for example, Flopamm™AN-132 and Flopamm™ AN-125 available from SNF. The difference between the first polymer fluid and the second polymer fluid (e.g., concentration, charge, molecular weight) should be sufficient to focus the tracer particles such that a sharp peak is obtained when the fluid is produced after shut-in. After the leading polymer bank, the trailing polymer bank, and the tracer particles have been introduced into the well, the previously described shut-in step occurs.

Due to the opposing nature of the first chemistry and the second chemistry, a chemical gradient between the leading polymer bank 504 and the trailing polymer bank 506 forms during the shut-in step. As a result of the chemical gradient in the well, the core/shell tracer particles migrate to a narrow region of the formation, as shown in FIG. 5B. In the embodiment depicted in FIG. 5B, the core/shell tracer particles experience more attractive forces from the leading polymer bank, and more repulsive forces from the trailing polymer bank. The forces that either attract or repel the core/shell tracer particles may be diffusophoretic or electrokinetic forces. Thus, the core/shell particles gather in a more localized fashion in the well. Due to the narrower range of where the core/shell particles are located, when the core/shell particles rupture to release the tracers, the tracer chemicals are released in a smaller area of the formation. When the tracers are later collected in the produced fluid, they breakthrough at the surface in a much narrower volume of fluid, which may improve the overall accuracy and precision of the data collected.

Embodiments disclosed herein may provide at least one of the following advantages. The compositions and methods described herein may allow for a single injection of all chemical tracer components, as opposed to conventional methods which generally require at least two different injection steps to introduce two separate tracers. Due to the designed polymer shell of the disclosed core/shell tracer particles, compositions and methods in accordance with the present disclosure may allow for the tracer chemicals to be released in a specific location of the well at a specific time. In conventional methods, the tracers are introduced into the well and immediately begin to diffuse. The disclosed compositions and methods may allow for the use of a broader range of tracer chemicals, as the disclosed method does not require chemical tracers to undergo a reaction to produce a second tracer.

The singular forms “a,” “an,” and “the” include plural referents, unless the context clearly dictates otherwise.

As used here and in the appended claims, the words “comprise,” “has,” and “include” and all grammatical variations thereof are each intended to have an open, non-limiting meaning that does not exclude additional elements or steps.

When the word “approximately” or “about” are used, this term may mean that there can be a variance in value of up to ±10%, of up to 5%, of up to 2%, of up to 1%, of up to 0.5%, of up to 0.1%, or up to 0.01%.

Ranges may be expressed as from about one particular value to about another particular value, inclusive. When such a range is expressed, it is to be understood that another embodiment is from the one particular value to the other particular value, along with all particular values and combinations thereof within the range.

While the disclosure includes a limited number of embodiments, those skilled in the art, having benefit of this disclosure, will appreciate that other embodiments may be devised which do not depart from the scope of the present disclosure. Accordingly, the scope should be limited only by the attached claims.

Thus, particular implementations of the subject matter have been described. Other implementations are within the scope of the following claims

Although only a few example embodiments have been described in detail, those skilled in the art will readily appreciate that many modifications are possible in the example embodiments without materially departing from the scope of the disclosure. Accordingly, all such modifications are intended to be included within the scope of this disclosure as defined in the following claims. In the claims, means-plus-function clauses are intended to cover the structures described as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112(f) for any limitations of any of the claims, except for those in which the claim expressly uses the words ‘means for’ together with an associated function.

What is claimed:

1. A single well chemical tracer composition comprising:
core/shell tracer particles having a core and a polymer shell, wherein at least two tracer chemicals are encapsulated in the core/shell tracer particles; and
an aqueous fluid.

2. The composition of claim 1, wherein the core is a porous core comprising a porous material and the at least two tracer chemicals.

3. The composition of claim 1, wherein the core is a liquid core comprising the at least two tracer chemicals.

4. The composition of claim 1, wherein the core/shell tracer particles have a particle size of less than one micron.

5. The composition of claim 2, wherein the porous core material is selected from the group consisting of poly(methylmethacrylate), poly(ethylcyanoacrylate), poly(butylcyanoacrylate), an aluminosilicate, a silicate, and combinations thereof.

6. The composition of claim 2, wherein the porous core comprises pores having an average pore size of from 1 to 50 nm.

7. The composition of claim 1, wherein the at least two tracer chemicals are selected from the group consisting of ethyl acetate, ethanol, n-propyl alcohol, isopropyl alcohol, and combinations thereof.

8. The composition of claim 1, wherein the core/shell tracer particles comprise up to 50 wt.% of the at least two tracer chemicals.

9. The composition of claim 1, wherein the polymer shell is a polyester or a polyamide.

10. The composition of claim 1, wherein the polymer shell comprises a thickness of from 1 to 50 nm.

11. The composition of claim 1, comprising up to 10 vol.% of the core/shell tracer particles.

12. A method of determining residual oil in a reservoir, the method comprising:
introducing a tracer fluid into a wellbore, the tracer fluid comprising core/shell tracer particles having a porous core and a polymer shell, wherein at least two tracer chemicals are encapsulated in the core/shell tracer particles;
maintaining the reservoir for a period of time such that the core/shell particle is ruptured and the at least two tracer chemicals are released into the reservoir;
producing a produced fluid from the reservoir;
measuring a quantity of the at least two tracer chemicals in the produced fluid; and
determining a residual oil content of the reservoir based on the measured quantity of the at least two tracer chemicals in the produced fluid.

13. The method of claim 12 further comprising:
prior to introducing the tracer fluid into the reservoir, introducing a first polymer fluid into the wellbore; and
after introducing the tracer fluid into the reservoir and before maintaining the reservoir, introducing a second polymer fluid into the reservoir such that a chemical gradient is created from the first polymer fluid to the second polymer fluid.

14. The method of claim 13, wherein the first polymer fluid comprises a first polymer concentration, wherein the second polymer fluid comprises a second polymer concentration, and wherein the first polymer concentration is different from the second polymer concentration.

15. The method of claim 12, wherein the core/shell tracer particles comprise a particle size of less than one micron.

16. The method of claim 12, wherein the porous core comprises poly(methylmethacrylate), poly(ethylcyanoacrylate), poly(butylcyanoacrylate), an aluminosilicate, a silicate, and combinations thereof.

17. The method of claim 12, wherein the polymer shell is a polyester or a polyamide.

18. The method of claim 12, wherein the polymer shell comprises a thickness of from 1 to 50 nm.

19. The method of claim 12, wherein the tracer fluid comprises up to 10 vol.% of the core/shell tracer particles.
